(19) United States
(12) Patent Application Publication
(10) Pub. No.: US 2015/0150725 A1
(43) Pub. Date: Jun. 4, 2015

(54) DEVICE FOR VIEWING AND FOR PROTECTION AGAINST OPTICAL RADIATION AND SOLID OR LIQUID SPRAY

(71) Applicant: Commissariat à l'énergie atomique et aux énergies alternatives, Paris (FR)

(72) Inventor: Hervé Piombini, Esvres Sur Indre (FR)

(21) Appl. No.: 14/408,982

(22) PCT Filed: Jun. 25, 2013
(86) PCT No.: PCT/EP2013/063201

§ 371 Date: Dec. 18, 2014

(30) Foreign Application Priority Data
Jun. 27, 2012 (FR) 1256115

(51) Int. Cl. A61F 9/02

(52) U.S. Cl. CPC A61F 9/022

(57) ABSTRACT

This device comprises for example two screens (20, 22) opaque to radiation and withstanding sprays, and suitable for preventing the radiation and sprays from reaching the eyes (4, 6) of a user, and a device (24) for acquiring an image of a scene (8) and restoring the image to the user, comprising at least one image-formation optical device (26) rigidly secured to the screen (20) and associated with an imaging device (28) for acquiring the image of the scene (8), and at least one restoration device (30) for restoring the acquired image to the user. According to the invention, this restoration device comprises a device for projecting the image of the scene into the eye of the user.

### TECHNICAL FIELD

The present invention concerns a device for observing and protecting against optical radiations, in particular artificial optical radiations that exceed permitted exposure limits, defined by standards, and against solid or liquid sprays.

This device may consist of spectacles for protecting the eyes against optical radiations and solid or liquid spray; and these spectacles may be either safety spectacles with sidepieces or goggles.

However, the device may also consist of a mask covering the face in order to protect this part of the body against optical radiations and solid or liquid spray.

“Optical radiations” means ultraviolet radiation, visible radiation and infrared radiation. The present invention applies in particular to the universal protection of workers vis-à-vis high-luminance sources that may be coherent (lasers) or incoherent (arc lamps of welding arcs) and vis-à-vis novel light sources such as multispectral laser sources, light emitting diodes, or LEDs, of the cold white light type, insolators, for example those that use ultraviolet radiation, and white lasers that have broad spectra.

These spectra render ineffective many spectacles for protection against lasers or spectacles that are based on coloured filters or multilayer filters.

The invention also applies to protection against any intrusion, in the eyes, of objects, for example metal swarf or projected particles, or spray of liquids.

The invention may also be suited to the assistance of partially-sighted persons who require an increase in ambient luminosity in order to see or distinguish objects as well as the environment of such objects.

By way of example of the sources mentioned above, FIG. 1 shows two spectra I and II of LED lighting systems of the cold white light type (X-axis: wavelengths λ in nm; Y-axis: relative units u.r.). And FIG. 2 shows the spectrum of the radiation emitted by a white laser (X- axis: wavelengths in nm; Y-axis: spectral power density (SPD) in μW/nm).

A Directive from the European Parliament moreover requires the introduction of protective measures for workers against artificial optical radiations which, at the present time, do not exist as individual protection for the eyes. In this regard, reference can be made to the following document:

Directive 2006/25/EC of the European Parliament and of the Council of 5 Apr. 2006 on the minimum health and safety requirements regarding the exposure of workers to risks arising from physical agents (artificial optical radiation).

The present invention falls in particular within the scope of individual and universal eye protection for all sources of coherent or non-coherent optical radiations that exceed the permitted exposure limits, indicated in the European regulations EN 60825-1 which relate to the safety of laser equipment, or in international regulations such as those that are given by the ICNIRP (International Commission on Non-Ionizing Radiation Protection).

### PRIOR ART

Current techniques for individual eye protection against lasers are based on the wearing of spectacles equipped with coloured filters.

A filter of this type functions by absorption in a specific spectral band related to the wavelength of the laser while affording vision of the scene observed by virtue of the spectral transparency region of the filter used.

In addition, it may happen that the filters used do not fulfil their protection function in the case of high light fluxes because of phenomena of saturable absorption. In this regard, reference can be made for example to the following document:

Laser damage tests and optical densities of laser goggles, J Hue, C Pelle, P Garrec, O Lartigue, F Baume and J L Rochas, Laser-induced damage in optical materials: 1999, Boulder, Colorado, USA, 4-7 Oct. 1999, Proc. of the SPIE, vol. 3902, pp 500-511.

It is also possible to use multilayer filters functioning in reflection but the spectral response thereof varies according to the angle of incidence and therefore according to the orientation of the spectacles when the head of the user moves.

By way of example, FIG. 3 shows two transmission spectra of known laser protection spectacles, for an HeNe (I) laser and for an excimer (II) laser (X-axis: wavelengths λ in nm; Y-axis: transmission T as %). And FIG. 4 shows the spectral transmission ST of a filter centred on 1064 nm according to the angle of incidence a expressed in degrees.

Moreover, the choice of protective spectacles that it is necessary to use with a radiation source is made (1) from data coming from this source, data that are not necessarily available, and (2) according to rules issuing from European standards that relate to the individual protection of eyes against laser radiation but do not deal with the case of multispectral sources or the case of continuous spectra.

A multispectral source may consist of several lasers having different wavelengths; this is the case for example of the juxtaposition of an excimer laser (functioning in the ultraviolet range), an argon laser (functioning in the visible range) and a YAG laser (functioning in the infrared range) in the same work area.

And the continuous spectra are for example the spectra of white lasers or those of LEDs, in particular LEDs that are used for lighting, or the spectra of plasma emissions such as electric arcs for example, which have a continuous spectrum and whose emission lines are characteristic of the materials present.

Furthermore, the calculations to be made for choosing the protective spectacles are often lengthy and tedious for a non-specialist, when reference is made to the exposure values permitted for the eye (see norm EN 207 or EN 208).

In addition, before using protective equipment, it is always necessary to check that this is appropriate to the radiation encountered: there is a risk for example of using the wrong spectacles in the case of multiple lasers, or using damaged spectacles. This is because the latter may for example comprise a delaminated layer, a split filter, scratches or impacts.

The documents FR 2 954 090 and FR 2 954 091 describe eye protection spectacles comprising image-restoration screens, disposed directly in front of the eyes of a user.

These screens are placed too close to the eyes to afford correct vision of the scene being observed.

### DISCLOSURE OF THE INVENTION

The aim of the present invention is to remedy the aforementioned drawbacks.

To do this, an opaque screen in a mask or two opaque screens in protective spectacles are provided and a device for acquiring the image of a scene and retrieving the image acquired for the user of the mask or spectacles is associated with this screen or screens.

The invention advantageously uses miniaturised optical components that are now conventional because of the development of mobile telephones in which a digital camera is integrated.

In addition, in the invention, use is advantageously made of techniques of the type that make it possible to produce spectacles intended for 3D video games, television or cinema, and comprising controllable electronic devices, of very low weight, which are inserted in the sidepieces of these spectacles.

Precisely, the subject matter of the present invention is an observation and protection device for protecting at least the eyes against optical radiations and solid or liquid spray, comprising:
at least one screen that is opaque to optical radiations and withstanding solid or liquid spray and which is suitable for preventing the optical radiations and solid or liquid spray from reaching at least the two eyes of a user, and
an acquisition and restoration device for acquiring an image of a scene and restoring the acquired image to the user.

When this acquisition and restoration device is supplied by a battery, an alert device is preferably provided to warn the user of the state of charge of this battery.

The acquisition and restoration device comprises:
at least one image formation optical device that is preferably provided with an autofocus system and is rigidly connected to the screen and associated with an imaging device in order to acquire the image of the scene, and
at least one restoration device for restoring the acquired image to the user.

The device that is the subject matter of the invention is characterised in that the restoration device comprises a device for projecting an image of the scene into the eye of the user.

The aperture of the image-formation optical device can be automatically controlled in order to manage the optical flow as well as the position by means of an autofocus system for affording close vision for the user.

According to a first particular embodiment of the invention:
the acquisition and restoration device comprises a single image-formation optical device that is rigidly connected to the screen, and a single restoration device that is associated with one of the two eyes of the user, and
the focal distance of the image-formation optical device is substantially equal to the focal distance of the eye with which the restoration device is associated.

The image-formation optical device is preferably associated with an image sensor of the CCD type that may have automatic gain control and management of its aperture diaphragm in order to adapt the level of illumination of the region observed exactly as needed for correct observation by the user.

According to a second particular embodiment of the invention:
the acquisition and restoration device comprises two image-formation optical devices that are rigidly connected to the screen and suitable for affording stereoscopic vision of the scene, and two restoration devices that are respectively associated with the two eyes of the user and cooperate with the two image-formation optical devices and the associated two imaging devices, in order to restore an image in three dimensions to the user, and
the quotient of the focal distance of each image-formation optical device and the distance between the two image-formation optical devices is substantially equal to the quotient of the focal distance of the corresponding eye and the interpupil distance.

The imaging device may comprise an optical fibre bundle (intended for the imaging).

Such a bundle is advantageous in the particular cases where the interfering optical flow may be greatly attenuated by optical fibre systems (glass fibres for transmissions in the deep ultraviolet, silica fibres for infrared radiations at 10.6 μm), or for protecting the user vis-à-vis solid or liquid spray or even for assisting a partially-sighted user.

In a variant, the imaging device comprises a digital camera that enables the system to increase or reduce the contrast of the image and therefore to manage the gain of the image (AGC: automatic gain control) and which may be provided, or not, with an optical filter suited to the wavelength or to a range of wavelengths in order to attenuate or limit or eliminate the optical flow coming from this spectral domain. In this case, the restoration device preferably comprises a display device.

The restoration device preferably also comprises a device for correcting the vision of the user, in order to obtain a clear image of the scene in the eye.

This correction device may be chosen from variable optical devices and MOEMSs, that is to say micro-opto-electromechanical systems.

The device that is the subject matter of the invention may also comprise an image intensifier for increasing the luminosity of the image, in particular to assist partially-sighted persons or persons having for example cataract problems.

An automatic focusing system of the type that is found in photographic apparatus may be added (triangularisation) so that the user always has a clear image of the region that he is observing.

Furthermore, it is possible to provide an alert system, either by display using a green or red diode for example, or by a buzzer giving the user the state of charge of a battery that can be used in the device so that this user does not suddenly find himself without vision of his environment.

### BRIEF DESCRIPTION OF THE DRAWINGS

The present invention will be understood better from a reading of the description of example embodiments given below, purely by way of indication and in no way limitatively, with reference to the accompanying drawings, on which:

FIG. 1 shows two spectra of LED lighting systems of the cold white type, already described,

FIG. 2 shows the spectrum of the radiation emitted by a white laser, already described,

FIG. 3 shows two transmission spectra of known laser protection spectacles, already described,

FIG. 4 shows the spectral transmission of a filter centred on 1064 nm according to the angle of incidence, already described,

FIG. 5 is a schematic partial front view of a first particular embodiment of the invention,

FIG. 6 is a schematic partial plan view of this first particular embodiment,

FIGS. 7 and 8 illustrate schematically and partially the compensation for certain visual defects in a variant of this first particular embodiment,

FIG. 9 is a schematic partial view of another variant of this first particular embodiment,

FIG. 10 is a schematic partial front view of a second particular embodiment of the invention,

FIG. 11 is a schematic partial plan view of this second particular embodiment,

FIG. 12 illustrates schematically and partially a variant of the first particular embodiment,

FIG. 13 illustrates schematically and partially a variant of a second particular embodiment, and

FIG. 14 illustrates schematically and partially another example of the invention.

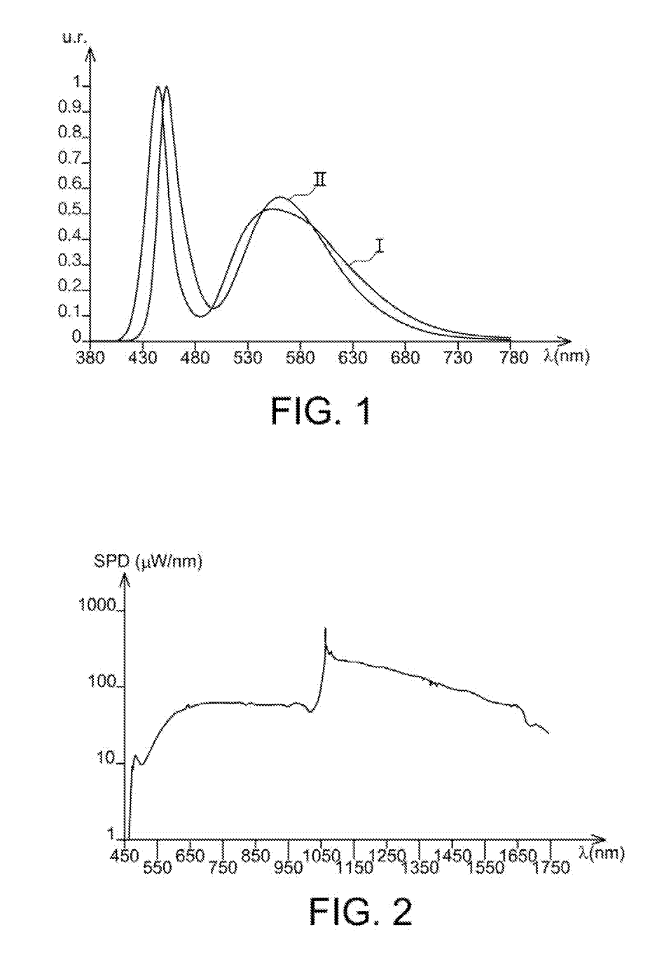

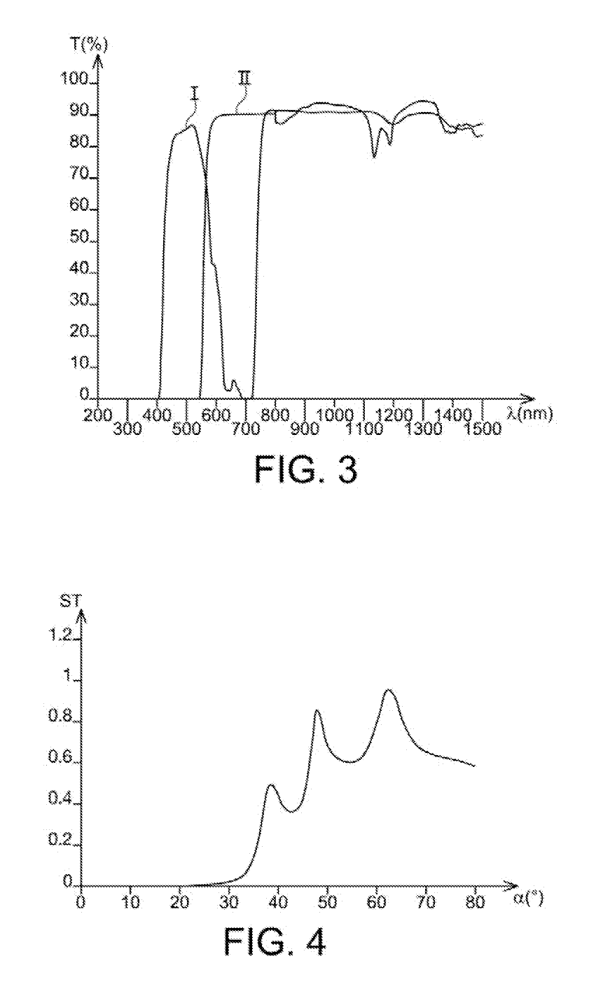

### DETAILED DISCLOSURE OF PARTICULAR EMBODIMENTS

Various examples of the device that is the subject matter of the invention are described below. In these examples, the device consists of spectacles for protecting eyes against optical radiations and solid or liquid spray.

The restoration of a scene to a user of these protective spectacles is effected by a device of the head-up display device type.

An image may thus be projected for one of the eyes of the user (or for each of his eyes) by means of a mini-mirror centred on the optical axis of the eye (or two mini-mirrors respectively centred on the optical axes of the eyes).

It is also possible to use more integrated devices comprising a set of prismatic arrays of the type developed by the company Optinvent.

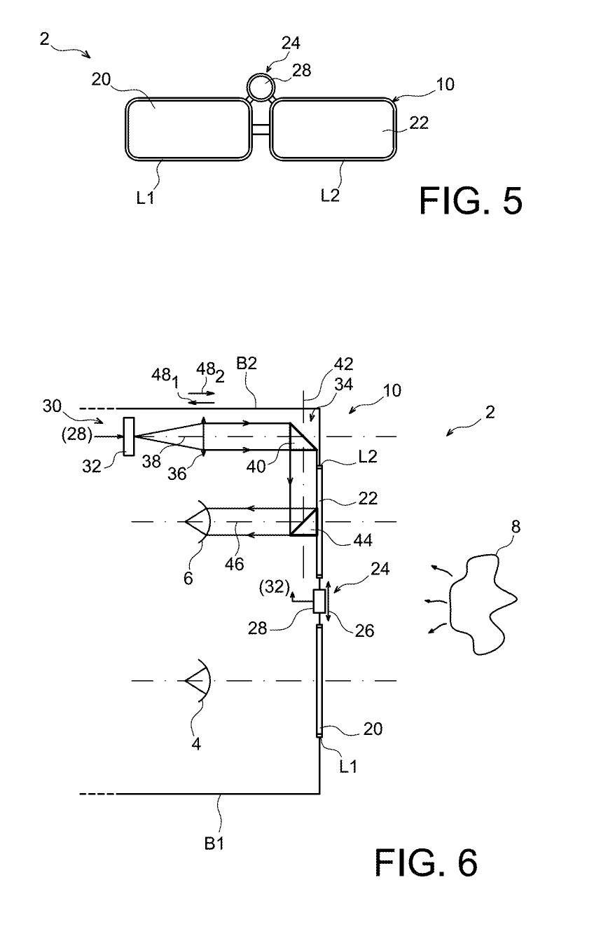

A first particular embodiment of the invention is illustrated by FIGS. 5 and 6.

FIG. 5 is a schematic partial front view of this first particular embodiment; and FIG. 6 is a schematic partial plan view thereof.

These FIGS. 5 and 6 show spectacles 2 intended to protect the eyes 4 and 6 of a user of these spectacles, in the process of observing a scene 8, against optical radiations and solid or liquid spray.

These spectacles are provided with a mask (not shown) that prevents lateral radiations or lateral spray from reaching the user.

The electrical supplies that are necessary for the functioning of the spectacles (by wire or battery) may be included in this mask.

The spectacles 2 comprise a frame 10 and, in accordance with the invention, they further comprise:
two screens 20 and 22 that are opaque to optical radiations and withstanding solid or liquid spray, are fixed to the frame and are respectively positioned so as to prevent the optical radiations and solid or liquid spray from reaching the two eyes 4 and 6 of the user, and
an acquisition and restoration device 24 intended to acquire an image of the scene 8 and to restore the acquired image to the user.

The two screens can be made from a self-coloured polymer (for example PMMA) or a refractory oxide or a metal, in particular a metal of the black oxidised aluminium type in the case of high-power lasers that may cause melting of the polymer or a cracking of the refractory oxide and therefore cause a risk to the user.

In the example depicted, the spectacles 2 also comprise two eyepieces L1 and L2 that are provided in the frame 10, and the latter is provided with two sidepieces that are represented by the lines B1 and B2, and the two screens 20 and 22 are respectively mounted in the two eyepieces L1 and L2.

In this example, the device 24 comprises:
an image-formation optical device 26 fixed to the frame 10, at the centre thereof, and forming part of a digital camera 28 forming an (active) imaging device for acquiring the image of the scene 8 with a good level of contrast by virtue of automatic adaptation of the camera gain or of the optical device opening, and
a restoration device 30 intended to restore the acquired image to the user.

This device 30 is associated with one of the two eyes of the user. In the example shown, it is the left eye 6.

In addition, the focal distance F_{c} of the optical device 26 of the camera 28 is substantially equal to the focal distance Fₒ of the eye 6, which is approximately 17 mm.

The digital camera 28, or the two digital cameras in the examples given below, is of course suited to the optical radiations in question.

With infrared radiation, it is possible to add digital mini-cameras for infrared in order to display the impacts of beams on the materials. With ultraviolet radiation, it is possible to add digital mini-cameras for ultraviolet in order to display the beam impacts on the materials. And with visible radiation, digital mini-cameras suited to such radiation are used; it is then possible to use black and white cameras or colour cameras.

Naturally the display screen or screens will restore a visible image of the scene observed.

The restoration device 30 comprises a display screen 32 that is electrically connected to the camera 28 and designed to display the image of the scene 8 that was formed in the camera 28 by means of the optical device 26.

The display screen, or the two display screens that will be dealt with below, can be chosen from liquid crystal display or LCD screens, plasma screens and organic light emitting diode or OLED screens.

The device 30 also comprises a device 34 for projecting an image of the scene 8 into the eye 6 of the user. This is because the image of the scene 8 is displayed in the direction of the latter by the display screen 32 and the eye 6 also looks in the direction of the scene 8.

As can be seen in FIG. 6, in the example described the projection device 34 comprises:
an image-takeup lens 36, the optical axis of which has the reference 38,
a prism 40 disposed on the axis 38 in order to divert at 90° the light issuing from the lens 36, along a (geometric) axis 42,
a mirror 44 at 45° in order to direct this light towards the eye 6 of the user, along the optical axis 46 of this eye.

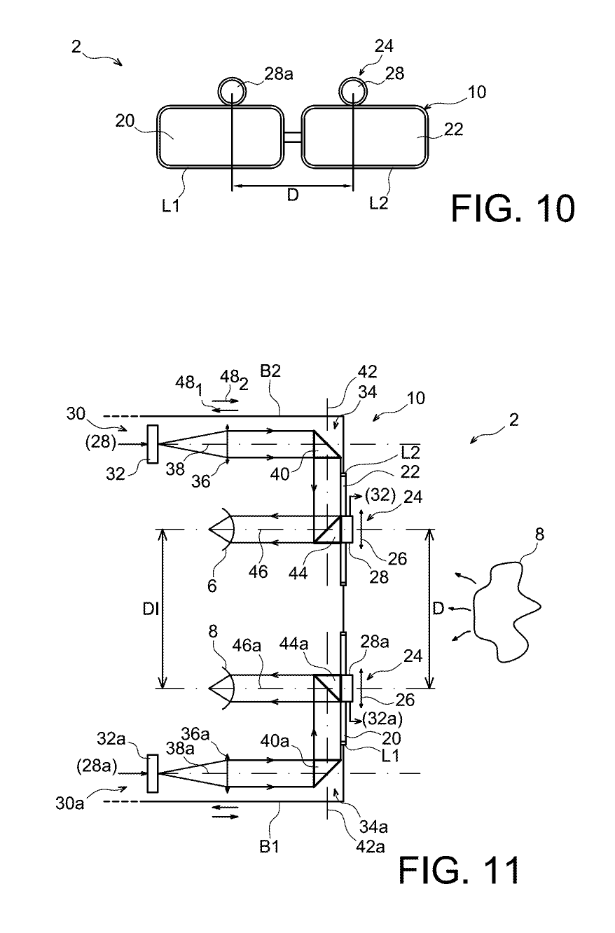

The mirror 44, or the mirrors if several of them are used to provide a reflection function (see in particular the example in FIGS. 10 and 11), may be of the dielectric type. This makes it possible to have the required image contrast according to the reflection coefficient chosen, and in particular to be able to obtain high efficiency close to 100%.

However, it is also possible to use simple metal mirrors because of the low cost thereof.

Preferably, the restoration device 30 also comprises a device designed to correct the vision of the user, in the case of myopia, hypermetropia or astigmatism thereof, in order to obtain a sharp image of the scene in the eye 6.

The lens 36 is then a suitable lens, single or multiple. It is wished for example to use an optical system of revolution that may be chromatic or not, according to the resolution required for the displayed image.

It is also possible to introduce a cylindrical lens of given power, the correction axes of which are oriented according to the relevant eye of the user with a view to correcting his astigmatism where applicable. Naturally, in the examples of the invention where each eye receives an image, as in the example of FIGS. 10 and 11, two such cylindrical lenses are used and each of them has its correction axes oriented according to the eye that is associated therewith.

In the example, the correction device is a variable optical device: the lens 36 is provided with means for moving it along its optical axis 38, means represented by the arrows 48₁ (in the case of a myopic user) and 48₂ (in the case of a hypermetropic user) in FIG. 6.

The prism 40 affords better stability of the image during movements of the observer (user) and preservation of the direction of the beam that is more or less independent of the position of the sidepieces of the spectacles.

It is also possible to provide means for rotating the lens 36 about its optical axis 38 in order to make corrections. If the protective spectacles are intended to be used by several persons, then a variable rotation of the lens 36 is provided so that each person can choose the astigmatism corrections that suit him, and a translation of this lens 36 in order to correct the various focusing defects related to the various persons (observers of scenes).

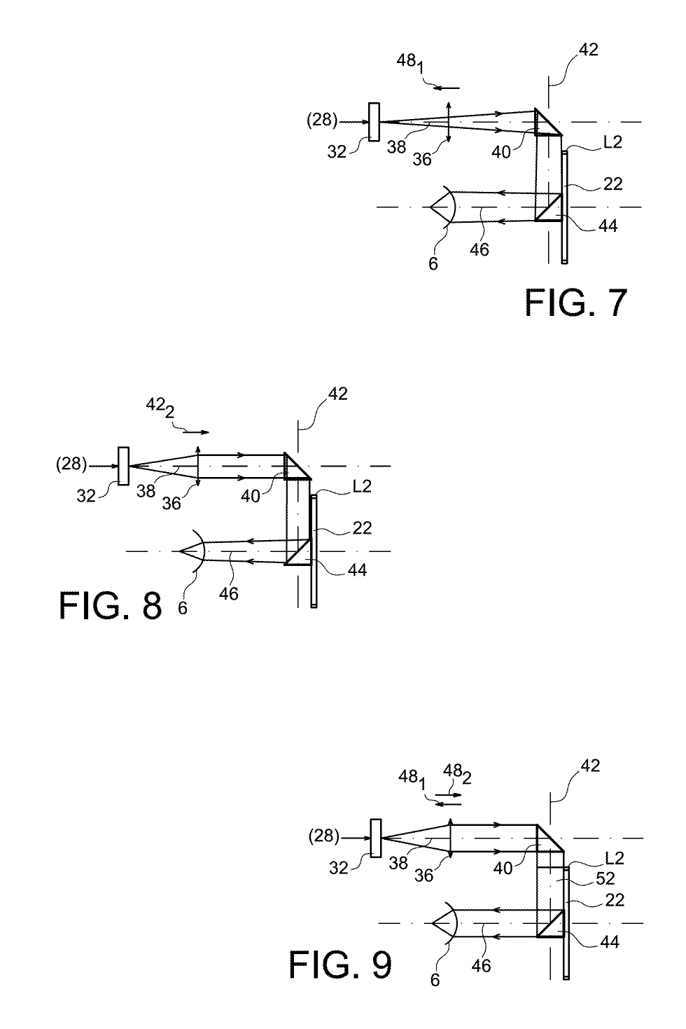

FIGS. 7 and 8 illustrate schematically and partially the movement of the lens 36 in order to compensate for certain visual defects of the user. In these figures, only certain elements in FIG. 6 have been repeated (with the same references).

The movement of the lens may be fixed, in the case where the protective spectacles are intended for a single user, or on the contrary adjustable in the case where these spectacles may be used by several persons.

FIG. 7 illustrates a correction in the case where the eye 6 is myopic. Then the lens 36 is moved closer to the display screen 32 in the direction of the arrow 48₁.

As for FIG. 8, this illustrates a correction in the case where the eye 6 is hypermetropic. Then the lens 36 is moved away from the display screen 32 in the direction of the arrow 48₂.

The visual corrections of the user (or users) mentioned previously may also be effected by means of MOEMS provided for this purpose.

FIG. 9 illustrates schematically and partially the possibility of replacing the mirror 44 in FIG. 6 by a prism 52 that works in total reflection and is designed to receive the light from the prism 40 along the axis 42 and to divert this light towards the eye 6 along the optical axis 46. In this figure, only certain elements in FIG. 6 have been repeated (with the same references).

A second particular embodiment of the invention is described below with reference to FIGS. 10 and 11.

FIG. 10 is a schematic and partial front view of this second particular embodiment; and FIG. 11 is a schematic partial plan view thereof.

As can be seen, in this second particular embodiment, the protective spectacles 2 again comprise the frame 10, provided with lens holders 12 and 14 and sidepieces 16 and 18; and the screens 20 and 22 are again mounted in lens holders 12 and 14 in order to protect the eyes 4 and 6 of the user, as explained in the description of FIGS. 5 and 6.

The difference between the two particular embodiments lies in the acquisition and restoration device 24 intended to acquire an image of the scene 8 observed by the user of the spectacles and to restore to him the image acquired: in the example in FIGS. 10 and 11, two digital cameras 28 and 28a are used instead of the single digital camera 28 in FIGS. 5 and 6, with a view to restoring a three-dimensional image of the scene 8 to the user.

The digital camera 28 corresponds to the left eye 6 of the user and the digital camber 28a to his right eye 4. In addition, the digital camera 28 is associated with the restoration device 30 that was previously described with reference to FIG. 6 and therefore uses the same components with the same references as can be seen.

As for the digital camera 28a, this is associated with another restoration device 30a. The latter is formed like the device 30 but, in order to distinguish the respective components of these devices 30 and 30a, those of the device 30a bear the references of the components of the device 30 followed by the letter a, as can be seen in FIG. 11.

The two imaging devices formed by the two digital cameras 28 and 28a are respectively provided with two identical image-formation optical devices 26 and 26a.

These lenses 26 and 26a can be controlled so as to manage the aperture thereof, making it possible to acquire more or less flux, or to manage the focusing thereof by means of an autofocus system. They may, where applicable, be provided with optical filters to protect the image sensor.

These lenses (and the associated digital cameras) are fixed to the frame 10, on either side of the centre thereof and so as to afford stereoscopic vision of the scene 8.

The two restoration devices 30 and 30a that are respectively associated with the two eyes 6 and 4 of the user thus cooperate with the two optical devices 26 and 26a and the associated two digital cameras 28 and 28a, in order to restore a three-dimensional image to the user.

The mirror 44 or the prism 52 are centred on the vision axis (or optical axis) of the user by mechanical movement (of the microscope binocular type) or by centring with respect to the frame, like a conventional spectacle glass, in order to comply with the pupil separation of the user.

Furthermore, the digital cameras are respectively centred on the optical axes of the eyes of the user if the focal length of the objection has the same focal length as that of the human eye, namely 17 mm. The relative positions of the cameras situated on the spectacles therefore depend on the interpupil distance DI, or interpupil separation, of the user and the focal lens of the object lens of the camera, so that the latter finds a perfect vision of the relief. The cameras therefore perfectly follow the movements of the head of the user.

The following condition is also complied with in order to preserve a good impression of the relief: the quotient of the focal distance F_{c} (or respectively F_{c}a of the image-formation optical device 26 (or respectively 26a) and the distance D (FIGS. 10 and 11) between the two optical devices 26 and 26a is substantially equal to the quotient of the focal distance Fₒ (or respectively Fₒa) of the corresponding eye 6 (or respectively 4) and the interpupil distance DI, which ranges from approximately 57 mm to 65 mm (FIG. 11).

In fact, in the example in question, the optical devices 26 and 26a are identical and therefore have the same focal distance F_{c}. Thus the above condition becomes: F_{c}/D substantially equal to Fₒ/DI and to Fₒa/DI. And, considering that the eyes have substantially the same focal distance Fₒ (which is equal to approximately 17 mm) and choosing D substantially equal to DI, the condition becomes: F_{c} substantially equal to Fₒ.

The adjustment of F_{c}/D to Fₒ/DI can be effected, for example, by machining the two opaque screens 20 and 22, or by a mechanical movement (of the microscope binocular type).

Naturally a vision correction device is preferably provided for each eye of the user, of the same type as the device described above: then means are provided for moving each of the two lenses 36 and 36a.

The prisms 40 and 40a afford better stability of the image during movements of the observer, and a preservation of the direction of the beam more or less independent of the position of the sidepieces of the spectacles.

Each of the mirrors 44 and 44a can be replaced by a prism of the same type as the prism 52, as explained above.

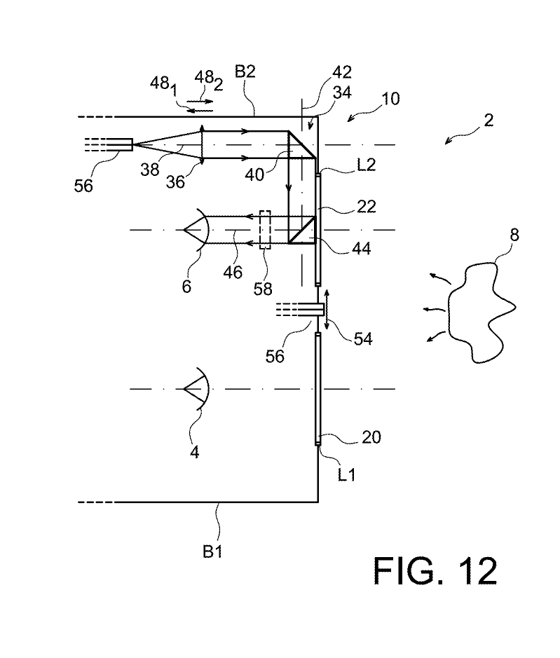

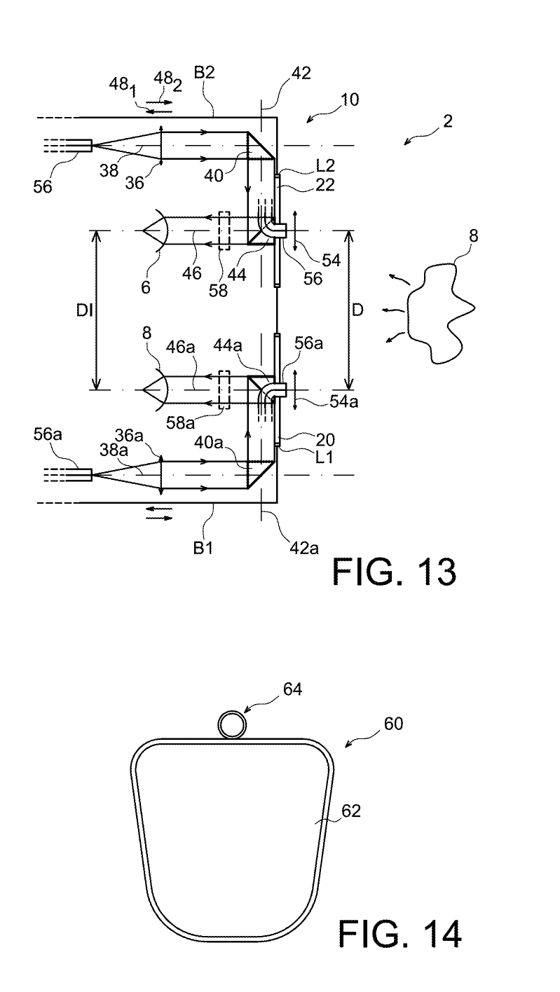

In the example in FIGS. 5 and 6, or in that in FIGS. 10 and 11, each assembly comprising a digital camera and a display screen can be replaced by an assembly comprising a bundle of optical fibres for (passive) imaging. This is illustrated schematically by FIG. 12, which corresponds to FIG. 6, and by FIG. 13 which corresponds to FIG. 11.

Such a passive system will be used only in the case where the bundle of fibres sufficiently stops, by intrinsic transmission of the material constituting the bundle of fibres, the radiation that is harmful for the eyes of the user.

In FIG. 12, an image-formation optical device 54 can be seen, corresponding to the optical device 26 of FIG. 6 and fixed, like the latter, to the frame 10, at the centre of this frame. A first end of a bundle 56 of optical fibres (for example made from glass, silica or plastic) is fixed to the frame 10, opposite the optical device 54.

This optical device 54 is designed so as to focus the light coming from the scene 8 onto the first end of the bundle 56. The second end thereof is fixed opposite the image takeup lens 36.

In FIG. 13, two image-formation optical devices 54 and 54a can be seen, corresponding respectively to the optical devices 26 and 26a of FIG. 11 and fixed, like the latter, to the frame 10, on either side of the centre thereof. A first end of a bundle of optical fibres 56 or 56a is fixed to the frame 10, on either side of the centre thereof. A first end of a bundle of optical fibres 56 or 56a is fixed to the frame 10, opposite the optical device 54 or 54a.

This optical device 54 or 54a is designed to focus the light coming from the scene 8 onto the first end of the bundle 56 or 56a. The second end thereof is fixed opposite the image takeup lens 36 or 36a.

Naturally, in the case in FIG. 12, the focal distance of the optical device 54, which can again be denoted F_{c}, is substantially equal to the focal distance Fₒ of the eye 6. Likewise, in the case of FIG. 13, the conditions mentioned above relating to the parameters denoted D, DI, Fₒ, Fₒa, F_{c} and F_{c}a apply here (with D: distance between the optical device 54 and 54a; and F_{c} or F_{c}a: focal distance of the optical device 54 or 54a).

If it is judged useful, a brightness amplifier 58 can be provided (FIG. 12) to increase the luminosity of the image before this image reaches the eye 6 of the user.

This brightness amplifier will in particular be reserved for protecting the eyes vis-à-vis the spraying of material or liquid. It can also serve for assisting vision for partially-sighted persons.

Likewise, two brightness amplifiers 58 and 58a can be provided (FIG. 13) for increasing the luminosity of the images before they reach respectively the eyes 6 and 4 of the user.

Such brightness amplifiers may even be used in the examples of the invention that use digital cameras.

In the examples given above, the light level can be adapted by acting on the gain of the digital camera or cameras, or on the intensity of the display screen or screens, or on the intensity of the brightness amplifier or amplifiers.

As seen, in the examples of the invention given above, the digital camera or cameras are fixed to the frame 10 of the safety spectacles. However, all the other components that equip these and make it possible to obtain an integrated vision device (in particular: bundle or bundles of optical fibres, prism or prisms, mirror or mirrors, lens or lenses, display screen or screens, optical device or devices), including the electronic devices and the electrical supply devices, such as batteries, which may be necessary to the functioning of the protective lenses, can be inserted in the sidepieces or in the opaque screens thereof, or be integrated in these sidepieces or opaque screens or, more generally, in the frame.

If it is judged useful, an autofocus system can be integrated in order to assist with the near vision of partially sighted users, having a cataract for example.

Where a total-reflection prism is used in association with one of the two eyes, or two total-reflection prisms respective associated with the two eyes, each prism may be placed in the rotation or fixing axis of the corresponding sidepiece of the protective spectacle so as to obtain an image that is insensitive to the defect caused by the fitting of the spectacles.

In this way protective spectacles provided with an integrated vision device is available.

It should be noted that some protective spectacles do not have sidepieces but are provided with lateral guards. In this case, the insertion or integration can be done in these side guards or at the latter.

The examples of the invention given above relate to protective spectacles that protect the two eyes of a user against optical radiation and solid or liquid sprays, while enabling him to observe a scene.

However, the invention is not limited to such spectacles: it is possible to design a device according to the invention consisting of a mask that protects the whole of the face of the person wearing this mask against optical radiations and solid or liquid sprays, while enabling him to observe a scene.

A person skilled in the art can adapt the examples given above to the production of such a mask, an example of which is seen in front view in FIG. 14, where it has the reference 60: this mask comprises a single screen 62 that protects the face and is opaque to radiations and withstands sprays; and this screen is also provided with a device 64 for acquiring the image of the scene and restoring the acquired image to the person wearing the mask.

Like the spectacles described above, the mask can be adapted to assist vision for partially-sighted persons.

What is claimed is:

1-9. (canceled)

10. Observation and protection device for protecting at least the eyes against optical radiations and solid or liquid spray, comprising:
at least one screen that is opaque to optical radiations and withstanding solid or liquid spray and which is suitable for preventing the optical radiations and solid or liquid spray from reaching at least the two eyes of a user, and
an acquisition and restoration device for acquiring an image of a scene and restoring the acquired image to the user,
wherein the acquisition and restoration device comprises:
at least one image-formation optical device that is rigidly connected to the screen and associated with an imaging device in order to acquire the image of the scene, and
at least one restoration device for restoring the acquired image to the user, wherein the restoration device comprises a device for projecting an image of the scene into the eye of the user.

11. Device according to claim 10, wherein:
the acquisition and restoration device comprises a single image-formation optical device that is rigidly connected to the screen, and a single restoration device that is associated with one of the two eyes of the user, and
the focal distance of the image-formation optical device is equal to the focal distance, about 17 mm, of the eye with which the restoration device is associated.

12. Device according to claim 10, wherein:
the acquisition and restoration device comprises two image-formation optical devices that are rigidly connected to the screen and suitable for affording stereoscopic vision of the scene, and two restoration devices that are respectively associated with the two eyes of the user and cooperate with the two image-formation optical devices and the associated two imaging devices, in order to restore an image in three dimensions to the user, and
the quotient of the focal distance of each image-formation optical device and the distance between the two image-formation optical devices is equal to the quotient of the focal distance, about 17 mm, of the corresponding eye and the interpupil distance which goes from about 57 mm to 65 mm.

13. Device according to claim 10, in which the imaging device comprises a bundle of optical fibres.

14. Device according to claim 10, in which the imaging device comprises a digital camera.

15. Device according to claim 14, in which the restoration device comprises a display device.

16. Device according to claim 10, in which the restoration device also comprises a device for correcting the vision of the user in order to obtain a sharp image of the scene in the eye.

17. Device according to claim 16, in which the correction device is chosen from variable optical devices and micro-opto-electromechanical systems.

18. Device according to claim 10, further comprising a brightness amplifier for increasing the luminosity of the image.
